(19) United States
(12) Patent Application Publication
(10) Pub. No.: US 2016/0178611 A1
(43) Pub. Date: Jun. 23, 2016

(54) THREE-DIMENSIONAL TRANSGLUTAMINASE-CROSSLINKED HYDROGEL FOR TUMOR ENGINEERING

(71) Applicants: Bo Han, Temple City, CA (US); Josephine Fang, Los Angeles, CA (US)

(72) Inventors: Bo Han, Temple City, CA (US); Josephine Fang, Los Angeles, CA (US)

(21) Appl. No.: 14/680,528

(22) Filed: Apr. 7, 2015

Related U.S. Application Data
(60) Provisional application No. 61/976,346, filed on Apr. 7, 2014.

(51) Int. Cl. G01N 33/50

(52) U.S. Cl. CPC G01N 33/5011

(57) ABSTRACT

Development of a physiologically relevant 3D model system for cancer research and drug development represents quite a challenge. We have adopted a 3D culture system based on a transglutaminase-crosslinked gelatin gel (Col-Tgel) to mimic tumor 3D microenvironment. The system has several unique advantages over other alternatives which include cell-matrix interaction sites provided by collagen derived peptides, a 3D construct suitable for reproducing the solid tumor microenvironment including multicellular tumor spheroids and metabolic gradients. In addition the controllable gel stiffness provides a wide range of mechanical restrictions; and compatibility with imaging based screening due to its transparent properties. In addition the Col-Tgel provides a cure-in-situ delivery vehicle for tumor xenograft formation in animals with a high take rate. Overall, this unique 3D system could provide a platform to accurately mimic in vivo situations to study tumor formation and progression both in vitro and in vivo, as well as for screening antineoplastic drugs and assessing the occurrence of drug resistance related to cancer cell stress.

### CROSS-REFERENCE TO RELATED APPLICATION

This application claims the benefit of U.S. Provisional Patent Application Ser. No. 61/976,346 entitled “THREE-DIMENSIONAL TRANSGLUTAMINASE-CROSSLINKED HYDROGEL FOR TUMOR ENGINEERING,” by Bo Han, Ph.D., filed on Apr. 7, 2014, the contents of which are hereby incorporated herein in their entirety by this reference.

### FIELD OF THE INVENTION

This invention is directed to the preparation and use of a transglutaminase-crosslinked three-dimensional tumor model for tumor modeling, including for use for screening and testing the efficacy of antineoplastic therapeutic agents.

### BACKGROUND OF THE INVENTION

Tumors are dynamic and complex structures. Their composition and environment are governed by biochemical and molecular signals exchanged between cells and their extracellular matrix (ECM) [1,2]. Even though 2D tumor cell cultures have been used routinely for conducting biochemical and drug sensitivity tests in oncology, they seldom mimic the in vivo environment, and scarcely reflect integral biomimetic characteristics such as cell-cell and cell-matrix interactions and their corresponding spatiotemporal signaling, metabolic gradients, and mechanical restriction [3-5]. Thus, developing bioengineered tumors by using biological relevant 3D tumor cell culture models can serve as a bridge between in vitro cell based assay and the native microenvironment of living organisms [6] [7-9]. In addition, 3D culture systems using human tissue could be a better tool for drug screening by providing more accurate in vivo like structure and organization and may produce a more predictive response than non-human systems [10].

Many 3D tumor cell culture models ranging from scaffold dependent to scaffold free, and consisting of single or multiple cell types have been developed. These models provide the opportunity to simulate important aspects of tumor masses including cancer cell aggregation and clustering, cell migration and proliferation, angiogenic factors release and hypoxia [8]. One of the most widely used models is the Multicellular Tumor Spheroids (MCTS) system, a scaffold-free tumor cell system that can facilitate cell-cell interactions through chemical linkers or gravitational enhancement [11-13]. Many extracellular matrices (ECM) such as Matrigel, type I collagen, fibrin, and hyaluronic acid have been used as tumor cell 3D scaffolds [14-18]. These biologically derived matrices provide both chemical and mechanical cues essential for changes in gene expression while allowing for cellular adhesion and integrin engagement [8,9]. However, there are still some unmet needs for cancer research and drug development, such as unknown amount of growth factors and additives in the preparations, uncontrollable mechanical rigidity, batch to batch variations, lack of reproducibility, difficult protocols, and non-native matrices for cells.

Therefore, there is a need for an improved three-dimensional model for tumors that is defined and reproducible and provides more accurate tumor modeling, especially with respect to the use of the model for assessing the effect of chemotherapeutic agents on tumors.

Additionally, carcinoma invasion and metastasis are under intensive investigation. Even though long been depicted as tumor cell autonomous phenotype due to gene mutation, recent studies point out that metastatic potential are acquired through exposure of epithelial cancer cells to the tumor microenvironment, triggered by paracrine signals, environmental stimuli, and ECM topologies. Although many clinical reports fostered the concept of transient EMT-MET switches in metastasis, there is only limited experimental proof. Recent animal studies support the role of an EMT in dissemination and the need of a MET for efficient metastasis (Tsai, Donaher et al. 2012). To find evidence to prove that human tumor cells goes from EMT at the primary tumor site before invasion come back to EMT at the metastatic sites is highly desired.

### SUMMARY OF THE INVENTION

The present invention provides an injectable gelatin-based transglutaminase-crosslinked three dimensional tumor model that meets these needs.

One aspect of the present invention is a composition of matter comprising a substantially insoluble three-dimensional matrix for tumor modeling wherein the three-dimensional matrix comprises gelatin covalently cross-linked by the catalytic action of transglutaminase forming covalent bonds between the acyl groups of glutamine side chains and the ε-amino groups of lysine side chains. The three-dimensional matrix can comprise from about 3% to about 9% of cross-linked gelatin. Typically, the three-dimensional matrix comprises about 3%, about 4.5%, about 6%, about 7.5%, or about 9% of cross-linked gelatin. Typically, the three-dimensional matrix is in the geometrical form of a plug or a dome. Preferably, the three-dimensional matrix is in the geometrical form of a dome. The three-dimensional matrix can further comprise at least one additional protein selected from the group consisting of collagen I, collagen II, collagen III, collagen IV, laminin, vitronectin, and fibronectin. Alternatively, the three-dimensional matrix can also further comprise at least one proteoglycan. In another alternative, the three-dimensional matrix can also further comprise at least one glycoprotein. Typically, the three-dimensional matrix is substantially transparent.

Another aspect of the present invention is a tumor model comprising:

(1) a three-dimensional matrix as described above; and

(2) a tumor cell line seeded into the three-dimensional matrix.

The tumor cell line can be any conventional human or non-human tumor cell line. For example, and not by way of limitation, the tumor cell line can be breast cancer cell line MDA MB-231, breast cancer cell line MCF-7, colon cancer cell line HCT116, Saos-2 human osteocarcoma cell line, C4-2B human prostate cancer cell line, SCC-71 human oral squamous carcinoma cell line, and colon cancer cell line CFPAC-1.

The tumor model can further comprise at least one additional cell line seeded into the three-dimensional matrix, such as, but not limited to, a mesenchymal cell line.

Yet another aspect of the present invention is a method for screening for the activity of a drug for antineoplastic activity comprising the steps of:

(1) providing a three-dimensional matrix as described above;

(2) providing a tumor cell line;

(3) seeding the tumor cell line into the three-dimensional matrix;

(4) culturing the tumor cell line in the three-dimensional matrix to provide a tumor culture in a three-dimensional environment;

(5) adding a quantity of a drug to be tested for antineoplastic activity to the tumor culture in the three-dimensional environment; and

(6) determining the effect of the drug on the viability of the tumor cell line in the three-dimensional environment to screen the drug for antineoplastic activity.

Various methods for determining the effect of the drug on the viability of the tumor cell line in the three-dimensional environment can be used. The drug to be tested can be a drug of any class of drugs with potential antineoplastic activity. For example, and not by way of limitation, the drug can be an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a camptothecin, an anti-tubulin agent, a thymidylate synthase inhibitor, a taxol derivative, or another class of drugs.

Yet another aspect of the present invention is a method of inducing tumor formation in an animal model for studying tumor growth in vivo comprising the steps of:

(1) providing a tumor model as described above; and

(2) implanting the tumor model into a test animal to study tumor growth in vivo.

This method can further comprise the steps of:

(3) administering a drug to be tested for antineoplastic activity into the test animal into which the tumor model has been implanted; and

(4) determining the effect of the drug on at least one parameter associated with tumor growth in the test animal.

The at least one parameter associated with tumor growth can be, but is not limited to, the rate or extent of tumor growth in the test animal as measured by tumor size or tumor burden, viability of the test animal, or activity or behavior of the test animal.

Yet another aspect of the present invention is a model for studying cell proliferation and migration. The model comprises the steps of:

(1) providing a tumor model as described above;

(2) monitoring cell proliferation within the model; and

(3) monitoring migration of cells out of the tumor model.

The rigidity of the transglutaminase gel can be controlled by gelatin concentration and cross-linking rate. Typically, cells migrate out of the tumor model driven by gel physical and chemical factors, such as rigidity, pH, O₂ level, the existence of binding sites for cell surface molecules, and other factors known in the art. The gel composite can be tailored. The cells that migrate out of the tumor model, which are not necessarily tumor cells, can migrate onto a plastic surface, or can be coated by ECM, collagen, or fibronectin. In the model, the three-dimensional structure, typically a dome, can be removed by a physical method, including, but not limited to, aspiration or scraping. The cells that have migrated out of the tumor model, which can be present on a hard surface, can be counted, or cell number can be determined by methods such as a MTT assay, a CCK-8 assay, or a bromodeoxyuridine (BrdU) assay.

Yet another aspect of the present invention is a method of generating a model of stressed tumor cells and employing the model to assess drug resistance. The method comprises the steps of:

(1) providing a tumor model as described above;

(2) subjecting the tumor model to a stress selected from the group consisting of: (i) mechanical restriction; and (ii) the buildup of at least one metabolite that results in chemical stress;

(3) administering an antineoplastic therapeutic agent to the tumor model that has been subjected to the stress;

(4) determining the degree of resistance to the antineoplastic therapeutic agent administered to the tumor model that has been subjected to the stress; and

(5) comparing the degree of resistance to the antineoplastic therapeutic agent occurring in the tumor model that has been subjected to the stress to the degree of resistance to the antineoplastic therapeutic agent occurring in a culture of tumor cells that are the same as in the tumor model but that have not been subjected to stress in order to determine the degree of resistance to the antineoplastic agent induced by stress.

Typically, the degree of resistance to the antineoplastic therapeutic agent is determined by a reduction in apoptosis induced by the antineoplastic therapeutic agent. The antineoplastic agent can be, but is not limited to: an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a cam ptothecin, an anti-tubulin agent, a thymidylate synthase inhibitor, or a taxol derivative. Particular frequently used antineoplastic agents for which resistance can be determined include taxol, etoposide, doxorubicin, cam ptothecin, and vincristine.

### BRIEF DESCRIPTION OF THE DRAWINGS

The following invention will become better understood with reference to the specification, appended claims, and accompanying drawings, where:

FIG. 1, shown as FIGS. 1A, 1B, 1C, 1D, 1E, and 1F is a schematic illustration of the Col-Tgel tumor model. FIG. 1A shows enzymatic crosslinking of collagen base gel by transglutaminase. FIG. 1B shows tumor cells suspended in soluble gel at desired densities. FIG. 1C shows gel was pipetted into a single well as a droplet, or multi droplets in a single dish after addition of crosslinker, Tgase. FIGS. 1D and 1E show comparison of transparency of three different 3D matrices, Type I collagen, Matrigel, and Col-Tgel. FIG. 1F shows the stiffness of Col-Tgel was manipulated by changing the gel concentrations.

FIG. 2, shown as FIGS. 2A, 2B, 2C, 2D, and 2E, is an illustration of 3D in vitro tumor models. FIG. 2A shows dome model, 20 μL of gel was pipetted on the bottom of each well of 48-well plate. FIG. 2D shows plug model, 100 μL of gel was added into each well of 96-well plate. MTT diffusion and reduction by MDA-MD-231 cells were monitored by inverted light microscope after 3 hours addition. Micrographs were obtained under light microscope on the edge (FIG. 2B) and center (FIG. 2C) of dome model; or surface (FIG. 2E) and bottom (FIG. 2F) of Plug model; B & C with 40× magnification, E&F with 100× magnification.

FIG. 3, shown as FIGS. 3A, 3B, 3C, 3D, 3E, 3F, 3G, and 3H, is an illustration of the formation of tumor spheroids in 3D gel. FIG. 3A shows breast cancer cells (MDA MB-231) were cultured in 3D dome gel for 6 days and formed spheroids inside the gel. Cells started to migrate out of gel either as individual cell or as collective cell clusters. Migrated cells showed elongated branched morphology on 2D surface. FIGS. 3B, 3C, 3D, and 3E show time course of spheroid formation (scale bar=140 μm). MDA MB-231 M cells were cultured in 3D Col-Tgel for 0, 2, 4, and 6 days and recorded observation under light microscope (FIGS. 3B, 3C, 3D, and 3E, scale bar=140 μm). FIG. 3F shows prostate cancer C4-2B cells; FIG. 3G shows colon cancer HCT116 cells; and FIG. 3H shows breast cancer MDA MB-231 cells were stained with F-actin (red) and DAPI (blue) and observed under fluorescence microscope (scale bar=100 μm).

FIG. 4, shown as FIGS. 4A, 4B, and 4C, shows that the 3D tumor model reproduces tumor cell heterogeneous conditions. FIG. 4A shows tumor cells formed quiescent or necrotic center, small to big spheroids from the innermost to the periphery region of the gel when MDA-MB-231 cells were cultured for 6 days (scale bar=1000 μm). FIG. 4B shows MDA MB-231 cells stained with proliferation marker anti-Ki67 antibody at day 6 and showed strong to faint staining from edge to center (Scale bar=300 μm). FIG. 4C shows oxygen concentrations were measured in the medium and in the gel. OM-1 oxygen meter probe was used to directly measure the O₂ percentage in medium and in gel. *P<0.01, data mean±standard deviation, n=4.

FIG. 5, shown as FIGS. 5A, 5B, and 5C, shows that tumor cells and mesenchymal cells showed different cell-cell and cell-matrix interaction in the 3D Col-Tgel system. FIG. 5A shows human squamous cell carcinoma line, SCC-71 and bone marrow derived mesenchymal stem cells were cultured separately or co-cultured together in the 3D Col-Tgel system. Top row, light microscope; bottom row, H&E staining from paraffin section. FIG. 5B shows MMP activity by Zymograph from conditioned medium of individual and co-culture. FIG. 5C shows cell viabilities in different culture conditions was by testing CCK-8 viability kit for dehydrogenase activity. Left: MDA-MB-231/MSC co-culture in 3D at initial 1:1 cell ratio; Right: SCC/MSC co-culture in gel in 3D at initial 1:1 cell ratio **P<0.05, data mean±standard deviation, n=3.

FIG. 6, shown as FIGS. 6A and 6B, shows the results of a drug sensitivity test on MDA MB-231 cells in 2D and 3D. FIG. 6A shows MDA MB-231 cells were treated with paclitaxel at concentrations of 0, 0.2, 2, and 20 μM (from left to right, scale bar=2000 μm) and analyzed with Live/Dead cytotoxicity/viability kit. Dead cells were in red and live cells in green. FIG. 6B shows MDA MB-231 cells displayed a dose response to paclitaxel in 2D and 3D. IC50 was 1.897 μM for 2D monolayer culture and 7.318 μM for 3D culture. ** P<0.05, data mean±standard deviation, n=3.

FIG. 7, shown as FIGS. 7A, 7B, 7C, 7D, 7E, 7F, 7G, 7H, and 7I, shows xenograft tumor induction using Col-Tgel as a carrier. FIG. 7A shows tumor cell and Col-Tgel mixture was injected at the desired site before gel curing. FIG. 7B shows initial tumor formation at the subcutaneous injection site 7-days post injection with gel border clearly defined (scale bar=2 mm). FIGS. 7C and 7D show: Osteosarcoma SaoS-2 cells, 200 μL of Col-Tgel with 1.0×10⁶ cells (dark arrow) or 100 uL of Col-Tgel with 0.5×10⁶ cells (red arrow) were injected subcutaneously in animals (scale bar=10 mm). Gross tumor formation by observed at day 3 (FIG. 7C) and day 20 (FIG. 7D). FIG. 7E shows retrieved tumors at day 20 exhibited positive correlation between tumor size and initial injection volume (scale bar=10 mm). FIG. 7F shows histological micrograph of Osteosarcoma tumor with vascular invasion (scale bar=180 μm). FIGS. 7G and 7H show MDA MB-231 cells formed clusters (green arrows) and blood vessel infiltration (hollow arrows) inside the Col-Tgel after 14 days of injection (FIG. 7G, scale bar=550 μm). MDA MB-231 cells (green arrow), host cells (blue arrows), and new blood vessels (hollow arrow) in the microenvironment created by co-delivery of tumor cells with Col-Tgel after 14 days of injection (FIG. 7H, scale bar=180 μm). FIG. 7I shows tumor formation curves of different cancer cells using Col-Tgel as carrier (MDA MD-231, HCT116 and CFPAC-1, scale bar=10 mm). The plot is a representative value of six tumors.

FIG. 8, shown as FIGS. 8A, 8B, 8C, 8D, and 8E, is an illustration that shows that 3D tumor model replicates tumor cell heterogeneous conditions. FIG. 8A shows hydrogel formation of Tg crosslinked gelatin. FIG. 8B shows cell and gel were mixed homogeneously at room temperature and delivered as a half dome shape into single well or multiple domes in one dish (FIG. 8C & FIG. 8D). Crosslinking of gelatin. Tumor cells formed necrotic or apoptotic center, small to big spheroids from the innermost to the periphery region of the gel when HCT116 cells were cultured in the gel for 6 days (FIG. 7E). Cell density was 4×10⁶/mL of gel, each dome tumor was 204 of gel. Scale bar=1000 μm.

FIG. 9, shown as FIGS. 9A and 9B, is an illustration showing that cell morphology and cluster formation is different at different gel conditions. FIG. 9A shows light view and fluorescence view. FIG. 9B shows graph showing cluster formation at 2 days and 6 days at gel concentrations of 3%, 4.5%, and 7%.

FIG. 10 shows that for a prolonged breast cancer MDA-MD-231 culture (>6 day), cell migration was observed at the edge the gel in the selected gel conditions.

### DETAILED DESCRIPTION OF THE INVENTION

An injectable three-dimensional tumor model based on transglutaminase-crosslinked gelatin provides a more reproducible tumor model that is particularly useful in determining the effect of chemotherapeutic agents on tumors.

The enzyme transglutaminase is an enzyme that catalyzes the formation of a covalent bond between a free amino group, such as that present in a lysine residue of a protein, and the acyl group at the end of the side chain of the amino acid residue glutamine in a protein. The reaction catalyzed by transglutaminase can be used to form intermolecular cross-links in proteins to form generally insoluble protein polymers.

The ECM (extracellular matrix) plays important roles in supporting or even inducing tumorigenesis [2]. The most common extracellular matrix component present in the tumor microenvironment is collagen, which provides a scaffold for structural support. Meanwhile, tumor microenvironment collagen turnover is related to tumor progression and metastasis [19-21]. In previous studies, we have developed an injectable gelatin-based transglutaminase-crosslinked gel system (Col-Tgel) for cell culture and drug delivery [22-24]. Here we focus on the development and validation of a novel 3D culture system that mimics the tumor stromal environment by using Col-Tgel. We demonstrated that biocompatibility and 3D architecture of Col-Tgel were suitable for reproducing the solid tumor microenvironment; and it may offer a toolbox to study key events associated with tumor formation, progression, and metastasis and have potential as an antitumor drug testing platform [25,26].

Materials and Methods

Cell Culture

MDA MB-231 (human breast carcinoma), Saos-2 (human osteosarcoma), and HCT116 (human colorectal carcinoma) cell lines were obtained from ATCC (American Type Culture Collection, Manassas, Va.). The C4-2B human prostate cancer cell line was generously provided by Dr. M. Stallcup and SCC 71 human oral squamous carcinoma cell line was gifted by Dr. Uttam Sinha (Norris Cancer Center at USC) [27,28]. MDA MB-231, Saos-2, SCC-71 were first expanded in traditional 2D culture in DMEM, HCT116 in McCoy 5a, and C4-2B in RPMI1640 (Mediatech, VA), all with 10% fetal bovine serum (Lonza, Md.) supplement and 1% Penicillin/Streptomycin (Mediatech, VA). Rat bone marrow derived mesenchymal stem cells were prepared in our laboratory as described [29].

Gel Preparation and Characterization

Transglutaminase-crosslinked collagen hydrogels (Col-Tgel) were prepared as described previously [22]. Briefly, 12% gelatin (bovine skin type B 225 bloom, Sigma-Aldrich, MO) was prepared with 2×PBS and autoclaved for sterilization. 4° C. stored stock gel was liquefied at 37° C. and further diluted to 6% with dH2O. Diluted gel was handled at room temperature for all assays and cell embedding.

Light transmission of Col-Tgel, compared with type I collagen (3 mg/mL, BD Bioscience) and Matrigel (Phenol red free, BD Bioscience) was measured at wavelengths of 600 nm using a UV Visible spectrophotometer (Hitch U-3000). Individual gel was loaded to 1 mL curvet followed by gelation. The higher transparency value represents the lower transparency of the gel.

Mechanical tests were carried out using an indentation test. Gelatin gel with concentrations of 3, 4.5, 6, 7.5 and 9% was prepared and 3 mL was loaded in a glass tube sample holder. After the gel polymerized, the gel surface was marked as initial height followed by gently applied a 5.8 g stainless steel spheres with a diameter of 8 mm. The sphere was placed at the centre of the sample and the weight of the sphere cased the deformation to occur. The side view image of the deformation image was required by mounted camera with a reference ruler. However, the ratio of the height of sample and the distance of indentation was not less than 10% and the ratio of the lateral dimension of sample and contact radius was not higher than 12. Therefore, the half-space assumption and Hertz contact theory cannot apply in this circumstance [30,31]. The central deformation of the gel construct cannot be directly used to compute the Young's modulus, so we simplified to use displacement distance as an indicator for relative gel stiffness in this study.

3d-Col-Tgel Culture

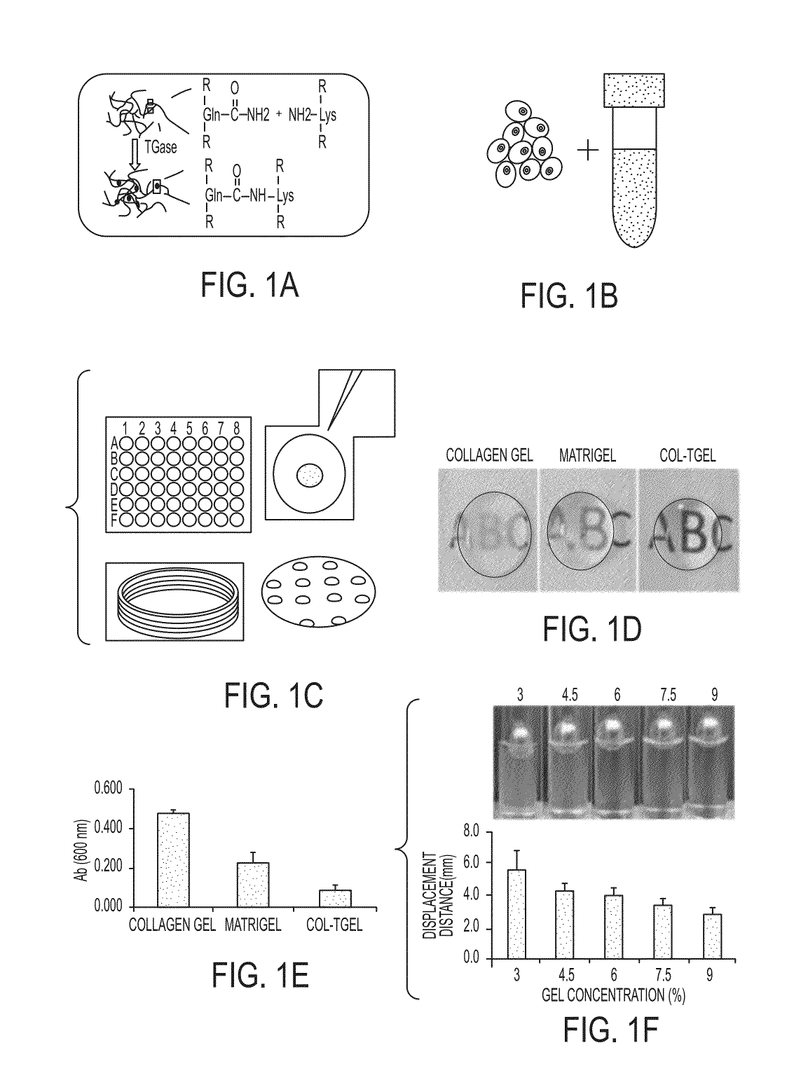

Cells were trypsinized (0.025% trypsin in HBSS, Mediatech), washed with 1×PBS and counted. A calculated volume of gelatin solution was added into the cell pellet to make desired final seeding density (FIG. 1). Cells were suspended in the gelatin solution followed by addition of 50 ul purified transglutaminase (Tg) crosslinker at a concentration of 1.308 mg/mL by gentle pipetting [23,32]. Cell-seeded hydrogel was placed as a single droplet on the surface of a 48-well suspension cell culture plate or multiple droplets on 4 mm dishes (Greiner bio-one, NC) followed by incubation at 37° C. for 30 minutes. The volume of the gel-cell suspension was varied to fabricate the specific radius of Col-Tgel-cell dome. Cell type specific medium was added for prolonged culture.

3D Morphological Analysis

Cells in 3D gel were directly observed daily under the light microscope (Leitz Wetzlar, Germany) and recorded with a digital camera (Nikon, Japan). The center-to-edge images were recorded using a light microscope by adjusting the focus plain on the z-stack.

For immunohistochemical analysis, cells in 3D gel were fixed with 10% neutral formalin for 10 minutes. After washing with PBS, cells were permeabilized with 0.5% Triton X-100/PBS, blocked with 10% FBS/DMEM for 30 minutes at 37° C. followed by incubation with 33 nM Rhodamine Phalloidin (F-actin staining) and 30 nM DAPI dihydrochloride (Biotium Inc., CA) (nuclei staining) in the dark for 30 minutes. Imaging was taken by EVOS fluorescence microscope (Advanced Microscopy Group, WA) directly in culture wells.

For non-fluorescence staining, the samples were proceed with same procedure as above and incubated with primary antibody Ki67 (dilution=1:100, Abcam, MA) and detected by Pierce Peroxidase Detection Kit (Thermo scientific, IL).

Cell Viability and Gel Permeability

Cell viability for the encapsulated cells was quantified by Cell Counting Kit-8 (Dojindo Laboratories, Japan) followed by manufacturer's protocol. Dehydrogenerase from live cells reduces red tetrazolium component into a soluble orange formazan in the medium. Briefly, at predetermined time points, media was replaced with 200 μL of fresh media containing 5 μL of stock CCkit8 reagent. After three hours of incubation, 100 μL of medium was transferred into a 96-well plate for 450 nm absorbance measurement by multiplate reader (Molecular Devices, CA). Medium without cell incubation served as baseline control.

For viability assay and gel permeability tests, cells embedded in Col-T gel were treated with 5 mg/mL of 3-(4, 5-dimethylthiazol-2-yl)-2,5-diphenyltetrazolium bromide (MTT) for 3 hours. The reaction was stopped by adding DMF/SDS (pH 4.7). The reduction of an MTT tetrazolium component into an insoluble dark purple formazan by viable cells was imaged by light microscope.

Oxygen Level Detection

MDA MB-231 cells were embedded in a 100 μl Col-Tgel droplet at 1×10⁶/mL and cultured in DMEM with 10% FBS and 1% PS at 37° C. Oxygen concentration was measured by OM-1 Oxygen meter (Microelectrodes Inc, NH) probed at the gel center and in the surrounding medium at day 0, 3 and 9.

Gelatinolytic Zymograph Assay

The culture medium was changed into serum free conditioned medium after 72-hour cell culture. Media or cell-gel samples were collected for gelatin zymography assay after another 24 hour incubation as described [33].

Cancer Drug Testing in a 3D Model

Each 20 μL gel droplet with 4×10⁴ cells was cultured in a 48-well-plate well for 3 days before drug treatment. Paclitaxel (TEVA Pharmaceuticals Inc., USA) in final concentrations of 0, 2, 20, and 200 μM were added. For comparison, the same numbers of cells as monolayer culture were treated with the same drug regime. After 72 hours, cell viability assay by CCkit-8 (Dojindo Laboratories, Japan) together with Live/Dead cell assay (Lifetech, NY) were performed. Briefly, 3D culture were incubated for 30 minutes in media containing 4 mM calcein AM (λem=530 nm) to stain viable cells and 1.5 mM ethidium homodimer-1 (λem=645 nm) to stain dead cells.

Tumor Induction in Nude Mice

All the animals were treated and housed according to approved IACUC (Institution of Animal Care and Use Committee at University of Southern California, permit number: 11732) guidelines and abided by the Guide for the Care and Use of Laboratory Animals. Total 36 of 6 week-old and ±22 g male athymic nude mice (Simonsen Laboratories) were used as xenograft hosts. Animals were divided into six groups for six type of cancer cell line induction. Briefly, 100 μL Col-Tgel containing 1×10⁶ cells of each tumor cell type (MDA-MB-231, C4-2B, HCT116, CFPAC-1, SaoS-2 and Calu-6) was subcutaneously injected into both flanks of mouse after anesthetizing with Ketamine/Xylazine. Tumor sizes were measured every three days. Retrieved tumor specimens were processed for pathological assays.

Results

The Collagen-Derived 3D Gel for Engineering Tumors

Col-Tgel contains native and denatured type I collagen peptides and provides a extracellular environment for cell attachment and growth [34] (FIG. 1A). Before crosslinking, gelatin maintains liquid phase at room temperature for homogenously mixing with cells (FIG. 1B). After adding of crosslinking enzyme, 3D gel could be formed in about 30 minutes at 37° C. This time window allowed to transfer gel-cell constructs to the culture plate as either a single or multi-gel droplets into each culture compartment (FIG. 1C). The dome height on the hydrophobic surface is correlated to the cast volume. After incubation, Col-Tgel transforms from liquid into irreversible hydrogel to encapsulate cells in situ. The cured Col-Tgel is transparent at all tested concentrations as compared to semi-opaque type I collagen gel and Matrigel, as shown by opaqueness comparison images (FIG. 1D) and light transmission measurement (FIG. 1E). This property enables to study live cell morphology change, cell-cell interaction, and migration possible even under the optical microscope. Moreover, Col-Tgel also exhibited variable mechanical stiffness property with the changes in gelatin concentrations and crosslinking rate. In general, the stiffness increases as the concentration of gelatin increased, where 9% Col-Tgel presented the highest stiffness while 3% was the lowest as illustrated by the indentation technique (FIG. 1F). This is an important property of the 3D gel, indicating gel restriction force would be directly manipulated by controlling the gel concentration to mimic interstitial pressure applied on cells.

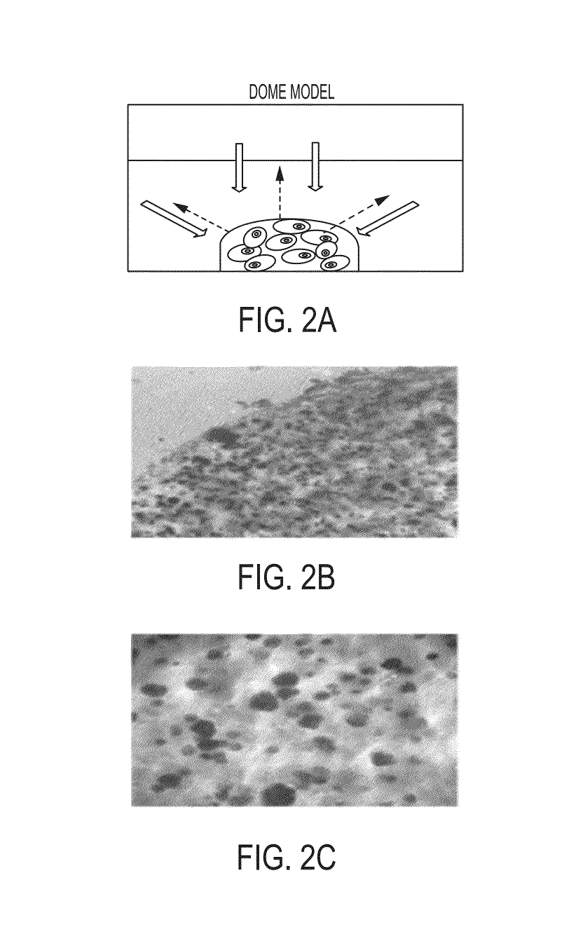

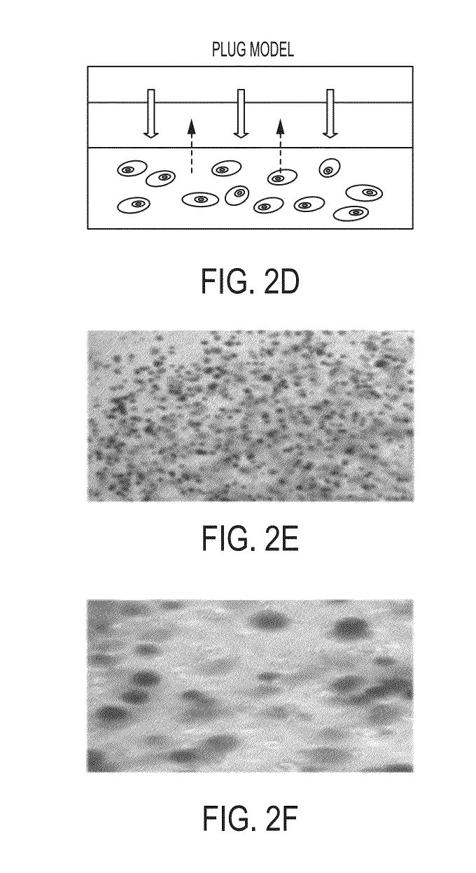

To further define the 3D culture system, we tested nutrient permeability and boundary effects on two types of 3D models: a dome model (FIG. 2A) and plug model (FIG. 2D). MTT was used as a sample molecule to study the model shape effects. Diffused MTT was absorbed by cells, and reduced into insoluble purple formazan crystals. After three hours, all MDA MB-231 cells in the dome model turned purple regardless of cell location (FIGS. 2 B&C). However, color changes in the plug model were cell location dependent. Cells changed from transparent to purple on the surface (FIG. 2E) while cells at the bottom still partially stained (FIG. 2F). In terms of geometry, the distances from center to margin were relatively more even in the dome shape than in the plug shape. Results suggest that a 3D dome model, without an initial nutrient diffusion barrier, was more suitable to study tumor progression than the plug model, therefore the dome model was selected for the succeeding studies.

Formation of Tumor Spheroids in 3D Gel

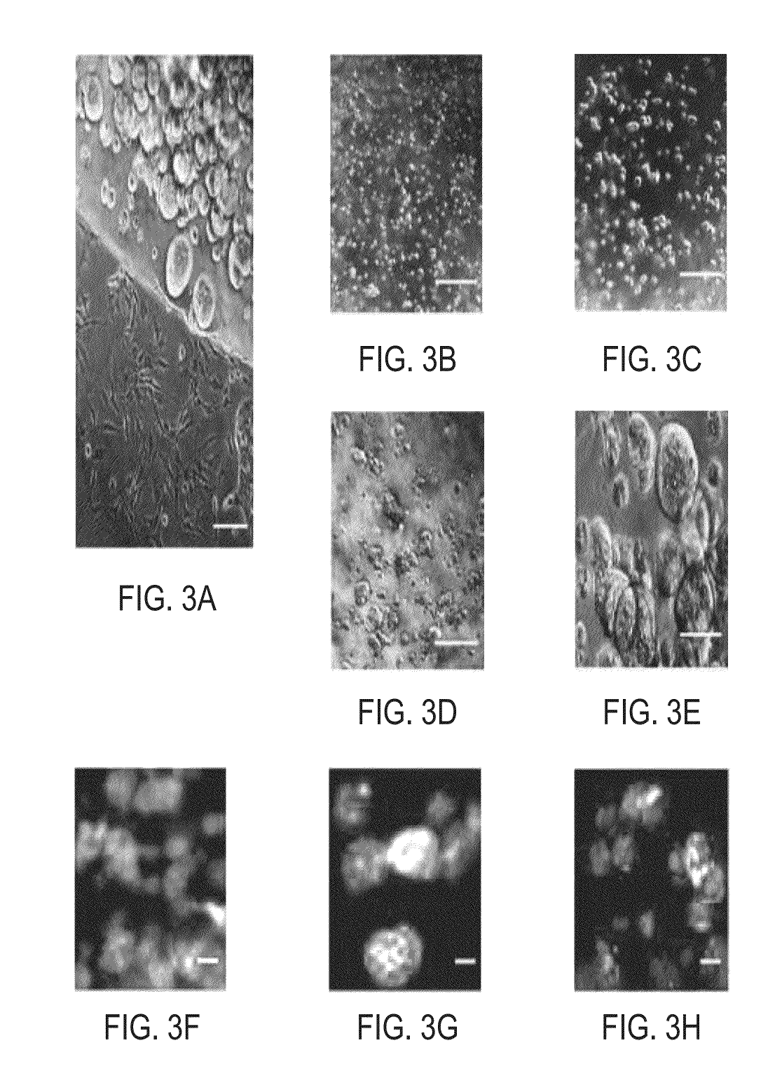

Col-Tgel serves as an interstitial substrate to support the growth of different cell types including tumor and normal cells. The transparency of Col-Tgel enabled us to observe cell assembly in real time. Tumor cells adopted unique morphology when cultured in Col-Tgel 3D in comparison to 2D plastic surface. MDA-MB-231 developed into spheroids inside Col-Tgel after 6 days of culture (FIG. 3A). Cells aggregated, formed tight cellular clusters ranging from 30 to 200 μm in diameter, and shaped similar to in vivo tumors (3A, top, inside gel). In comparison, the same MDA MB-231 cells growing on a plastic surface (3A, bottom half, on plastic) presented a completely different cellular morphology in the same culture well. Cells exhibited elongated morphology with pseudopodium protrusion. There was little cell-cell junction formation between neighboring cells.

The time course of spheroid formation is shown in (FIG. 3, B-E). On initial seeding, cells were homogenously distributed throughout the gel with no aggregates or clusters formation (FIG. 3B). After 48 hours, 2- to 3-cell aggregates formed (FIG. 3C). Using a time-lapse camera, initial cell clusters were formed by both cell multiplication and assembly (data not shown). After four days in culture, cell clusters grew bigger with 10 to 20 cells in each cluster (FIG. 3D). By six days, a large number of spheroids aligned around the periphery of the gel dome. Spheroids contained tens to one hundred cells and their shapes varied from round to oval with smooth surfaces (FIG. 3E). Other tumor cells such as prostate cancer cells (C4-2B), and colon cancer cells (HCT116) presented similar cell arrangements in 3D Col-Tgel (not shown). When stained with the cytoskeleton protein, F-actin, spheroids displayed robust cell-cell interactions in different tumor cells (FIG. 3F, prostate cancer C4-2B; 3G, colon cancer HCT116; and 3H, breast cancer MDA-MB-231) [35].

Heterogeneous Tumor Model Inducing Hypoxia

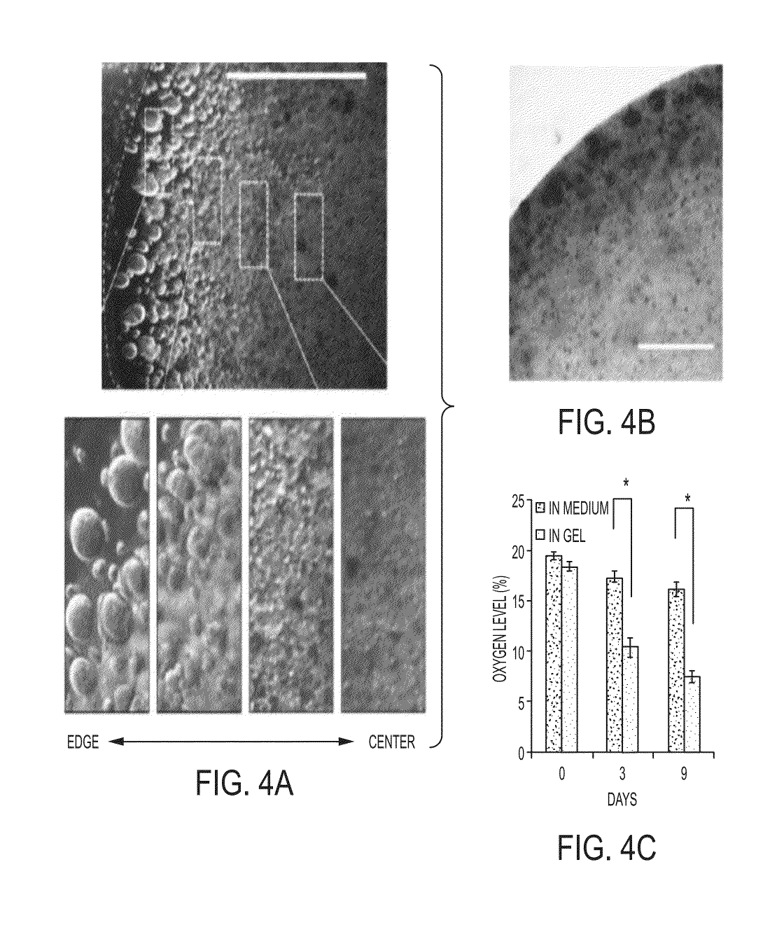

After culture in the 3D gel for a few days, cancer cells on the periphery were seen to actively proliferate and developed spheroids while the innermost ones remained FIG. 4 or necrotic (FIG. 4A and inserts). The sizes of tumor spheroids decreased from edge to center. When the distance is over 1000 μm from edge, the cells were less proliferative as shown by active proliferation marker Ki67 staining (FIG. 4B). A large number of dead cells presented in a large void with no visible cell structures. To examine if this phenomenon resulted from decreasing nutrient diffusion and depriving of oxygen at the dome center, we measured the oxygen concentrations using an oxygen probe. We found there was no difference in oxygen levels between gel and medium on the initial day. With increasing culture time, oxygen levels in Col-gel were lower than surrounding medium, and dropped significantly with the culture time (FIG. 4C). An hypoxia environment was established when actively proliferating cells acceleratively consumed oxygen, while in the meanwhile formed cellular barrier to prevent oxygen diffusion in the center. This feature presents an avascular and hypoxia tumor microenvironment.

Co-Culture of Cancer Cells and Mesenchymal Cells to Mimic Tumor Niche

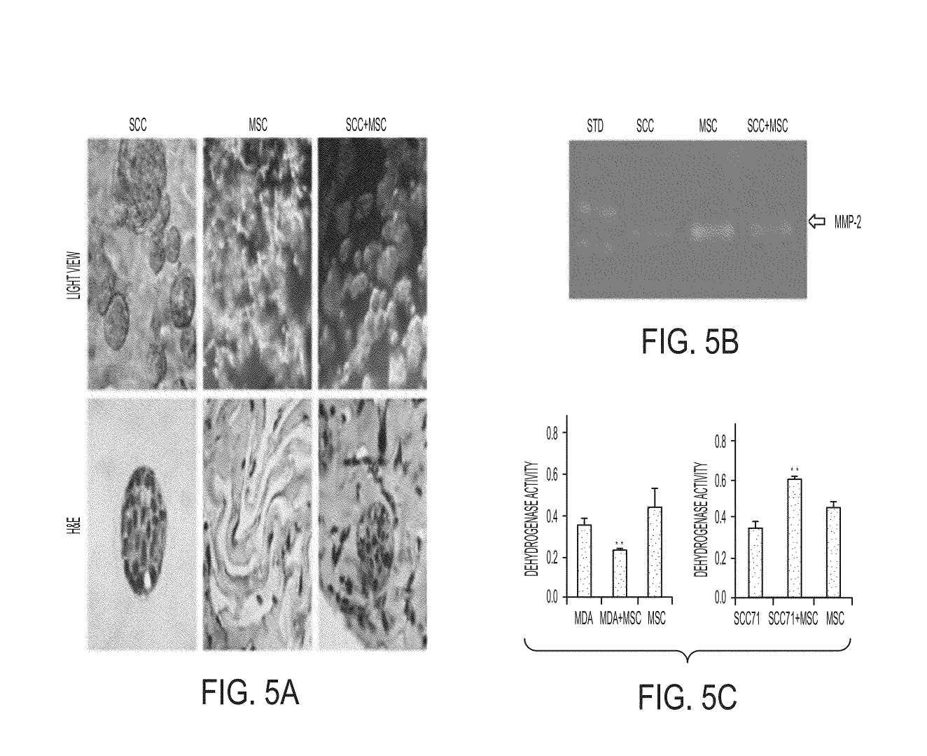

In vitro tumor models can be made more relevant by including two or more cell types to study cell-cell interactions and cell-matrix interaction. Human squamous cell carcinoma line, SCC-71 and bone marrow derived mesenchymal stem cells were cultured separately or co-cultured together in the 3D Col-Tgel system (FIG. 5A). SCC-71 cells formed spheroids in Col-Tgel after day 6. The multi-cell spheroids displayed borders in the gel without visible digestive rings. On the other hand, bone marrow derived mesenchymal stem cells exhibited another distinct morphology in the gel. After 6 days of culture, cells presented stellate structure and seemed to dig tunnels appearing with light reflection in the gel. H&E staining of MSCs showed cells were separated from each other with elongated shape and degraded the surrounding gel environment leaving multiple clefts. MMP activity by Zymograph from conditioned medium confirmed the results. MMPs activity in MSC was more active than in SCC-71 cell at the tested conditions (FIG. 5B). When SCC-71 cells were co-cultured with MSC cells at 1:1 ratio, both cells maintained their distinctive morphologies; SCC-71 formed clusters with tight junctions, while MSC cells kept as individual elongate shapes. However, MMP activity was altered in the co-culture condition. Unlike MSC alone cultures, co-cultures revealed apparent decreased ECM degradation by MSCs and decreased MMP-2 activity; indicating the paracrine signals exchanged among different cell types affected cancer cell behaviors. Beside morphology, we analyzed the cell viability (by testing dehydrogenase activity) in co-culture settings. Co-culture of SCC-71 and MSC showed increase in total dehydrogenase activity in comparison with single cell type culture (FIG. 5C). However, dehydrogenase activity decreased in MDA-MB-231/MSC co-culture system (FIG. 5C), suggesting environmental effect on tumor cell could be a dynamic process, depending on cell type and cell state.

3D Model for Drug Test

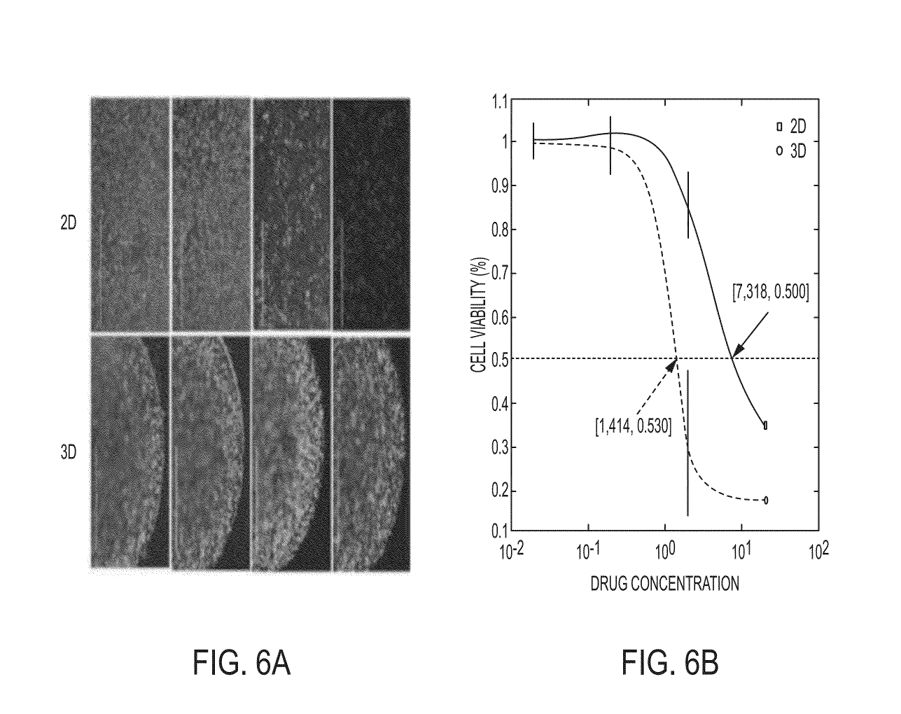

We further characterized the chemotherapy drug sensitivity in our 3D model. Taxol is a cytotoxic chemotherapeutic agent with proven clinical value in breast cancer. MDA-MB-231 cells cultured in 3D scaffolds demonstrated greater resistance than cells in 2D monolayer culture as shown by live/dead cell test (FIG. 6A) with IC50=1.897 μM for 2D and IC50=7.318 μM for 3D culture (FIG. 6B). Anti-mitotic drugs such as taxol were less effective in 3D culture at concentrations that were previously shown to cause apoptosis in monolayer culture.

Col-Tgel as a Carrier for Xenograft in Animal Model

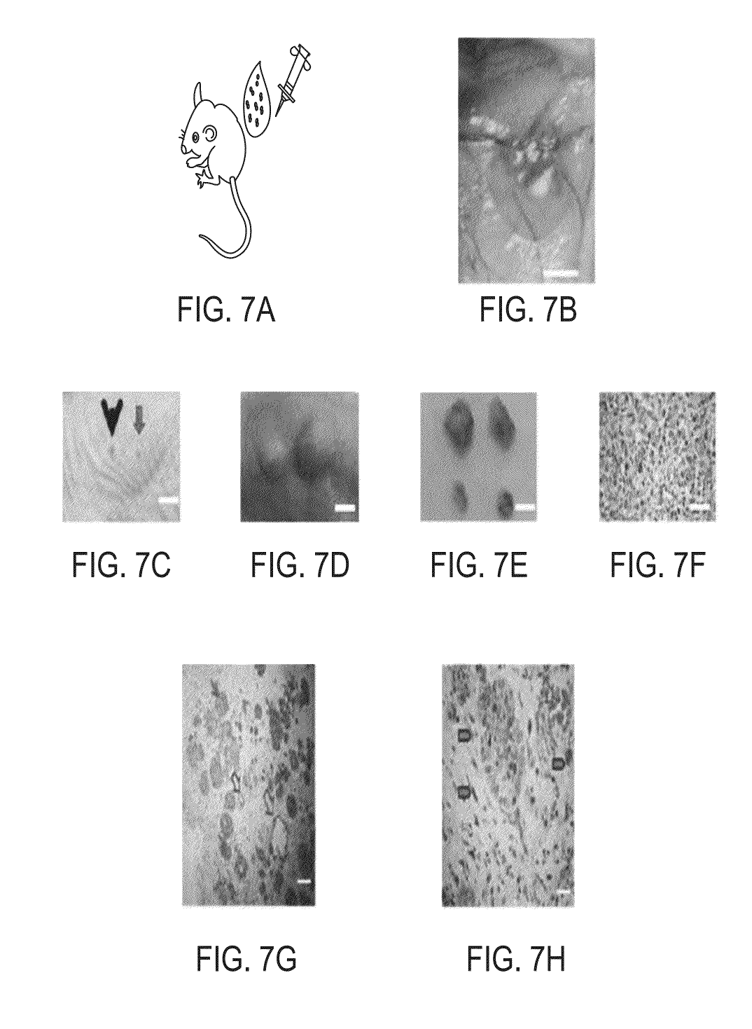

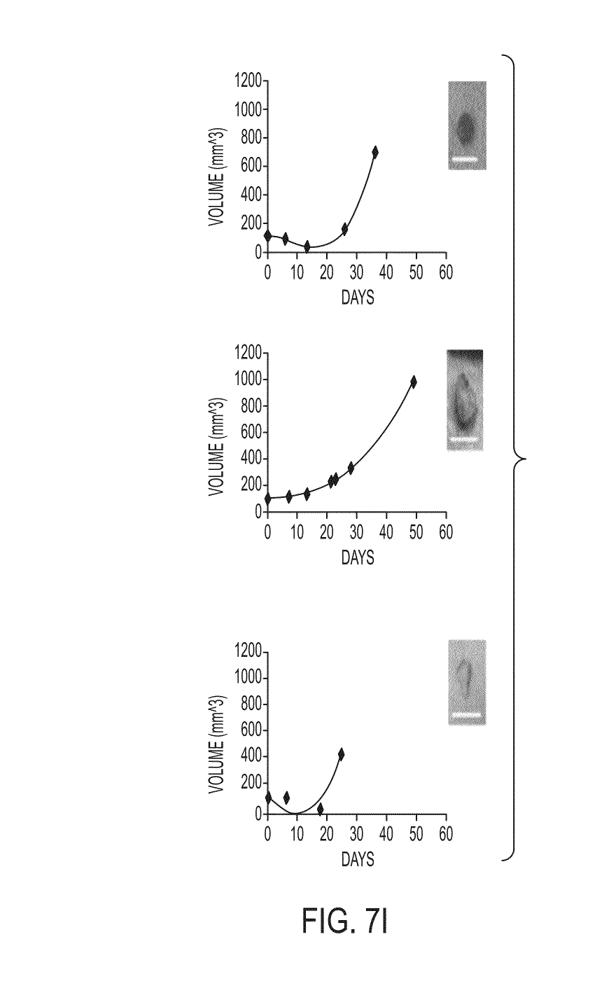

Xenograft tumors were induced using Col-Tgel as a carrier (FIG. 7A). Gel formed insoluble hydrogel subcutaneously at the injection site with tumor cells embedded. As shown in FIG. 7B, Gel structure was still clearly defined even after 7 days injection and blood vessels invasion was observed. Moreover, the size of tumors could be controlled by the injection volume of gel tumor (same cell density), where the osteosarcoma size by 200 μL of gel tumor was significantly larger than 100 μL after 28 days injection (FIG. 7 C-F). Two injection sides were close but tumor final masses did not fuse or intervene to each other. This observation suggested that the gel were capable of localized cell delivery. Importantly, early stage of tumor development and progression could be studied in the injected semienclosed gel environment. A histomicrograph of 7 day post injection of breast cancer cell MDA MB-231 clearly showed that tumor cells assembled into clusters ranging from 100 to 500 μm and disperse in the gel unevenly, where the center of gel had less clusters with smaller size while the peripheral region had larger and denser tumor cluster formation, similar to in vitro observations (FIG. 7 G-H). Interestingly, blood vessels clearly infiltrated into the gel at day 7 in response to tumor cells, providing nutrients together with host cells such as immune cells, inflammatory cells and mesenchymal stem cells to the tumor environment (FIG. 7H, hollow arrow and blue arrow). Tumor development induced by using Col-Tgel as a carrier was also tested for other tumor cells including breast cancer MDA MB-231, colon cancer HCT116, and pancreatic cancer CFPAC-1. Representative tumor formation curves were shown in FIG. 7I. Taken together, this gel carrier bioengineering tumor model may provide a tool box to study the orchestrated tumor formation and progression events including types of cell participation, spatiotemporal signals molecules exchange between cells, and extracellular matrix protein deposition and degradation.

### DISCUSSION

Col-Tgel is a tailorable collagen-based remodelable hydrogel system able to induce spheroid formation without the use of time and labor intensive protocols such as the hanging drop and linker-engineered method, sophisticated equipment like rotating wall vessel bioreactors, or special handling temperature to prevent self-assembly [11,12,36]. In vitro 3D culture systems to induce spheroid tumor formation by using synthetic, natural or hybrid materials have been extensively attempted [5,37,38]. Biocompatible materials such as agarose, methycellulose, PMMA, PEG are structurally suitable to provide support for tumor spheroid formation, however, they lack cell adhesion and enzyme cleavage sites like in vivo tissue [8,39-41]. On the other hand, 3D scaffold fabricated from ECM substrates such as Matrigel, type I collagen, laminin, and fibronectin are cell attachable and remodelable, however, they are unable to provide the wide spectrum of rigidity necessary to mimic normal and pathological in vivo conditions [8].

By using collagen peptides with an enzymatic crosslinking technique, Col-Tgel overcomes these limitations and thus appears as a suitable scaffold for in vitro and in vivo tumor engineering. Importantly, Col-Tgel offers handling flexibility through controlling the 3D construct size, shape, concentration, and crosslinking rate to achieve structural heterogeneity resemble in vivo tumor environment on many aspects, including nutrition diffusion and pH gradients, hypoxia environment, and mechanical restriction. Thus, by modulating these parameters according to tumor progression state, it is possible to bioengineer 3D tumor in vitro to closely resemble cancer cell growing in the in vivo environment.

We established imaging based assays based on transparent property of the Col-Tgel. Cell morphology change, delivery of chemotherapeutics, and cell migration and invasion could be monitored across 3D constructs in real-time by light or fluorescence microscope. Our results suggest that spheroid tumor in the Col-Tgel closely resemble in vivo avascular tumor nodular appearance and contained a necrotic core and outlayer proliferative rim, mimicking the highly preoperative in vivo tumor cells located near nutrient rich capillaries [42-46]. Overall, Col-Tgel 3D architecture presents physiologically relevant characteristics of tumor cells and it also features simple and easy operation protocols to examine multiple aspects of cancers.

Col-Tgel is able to be easily manipulated to implement wide range of stiffness by altering the gel concentration and crosslinking units [47]. A recent explosion of work shows the powerful influence of 3D biophysical properties, such as rigidity, porosity, density and geometry on cell fates [48-50]. Thus, by tailoring gel formulation, physical stiffness of tumor environment may be simulated at in vitro 3D setting to study cell proliferation, differentiation, apoptosis, senescence, and invasion behaviors [51-53]. To more accurately mimic tissue specific microenvironment, the matrix composition may also be altered by adding different type of extracellular matrix into Col-Tgel platform, such as various types of collagen (I-IV), adhesion molecules such as laminin, vetronectin, and fibronectin, and proteoglycans and glycoproteins [54-59]. For example, pancreatic tumor surrounds with dense fibrillar collagen while brain tumors composes a more amorphous matrix such as hyaluronic acid [60].

The solid cancer microenvironment is composed not only of tumor cells but also stromal cells. The tumor stroma consists of fibroblasts, adipocytes, inflammatory cells such as lymphocytes and macrophage and lymphatic and blood capillaries including pericytes and endothelial cells [61-64]. Cancer progression and metastasis depends on the crosstalk within the microenvironments [65-67]. However, tumor cells interaction of extracellular matrix, other cell types, or the immune system is scant or completely absent in the 2D monolayer culture. The 3D collagen-based Tgel (Col-Tgel) system provides a structure platform for spatial organization of tissues and cell-cell interactions. In our study, we tested co-culture of tumor cells with bone marrow meshenchymal stem cells in a 3D gel. Regarding cell morphologies, the H&E staining demonstrated that both cell types preserve their phenotypic traits. Cancer cells maintained their epithelial morphology and forming spheroid, whereas, MSC showed their typical spindle-shaped morphology. We also observed two type of cells formed chimera spheroids when cells were labeled with different fluorescence probes (Data not shown), the exact cause and effect of such interactions still needs to be elucidated. In fact, total cell viability and MMP expressions were significantly altered in the co-culture system as compared with single cell type culture. Thus, by using co-cultures, it is possible to recreate some of the in vivo tumor niches under highly controlled and reproducible fashions to study tumor cell morphology, phenotype, metabolism and invasion in vitro. Interestingly, we also observed host cells infiltration into the 3D gel system when delivering cancer cell for xenograft tumor induction. Col-Tgel forms a semi-enclosed system to prevent cancer cell diffusion, in the meanwhile, the cured gel acts as extracellular matrix to support surrounding cells infiltration and migration as they responding to the tumor cell signals. As a result, multicellular tumoroids are formed in situ followed by ECM remodeling at the injection site. We observed that angiogenesis occurred within 7 days, tumor nodular formation within 14 days and mature tumor development in 21-28 days. We anticipate this new bioengineering tumor will provide us an opportunity to study early stage host cell response by characterizing spatial temporal events of host cell populations and signals exchange, to gain insight of cell-cell communicate on and their contribution to tumor progression.

In conclusion, we developed a new bioengineering tumor model by using 3D ECM derived peptides in multiple well plates. It is easy to reproducible and cost-effective. The 3D model recapitulates key features of tumors in vivo including cell-cell interaction, cell-matrix interactions, and multicellular architecture. This technology allows creating uniform and highly-reproducible bioengineering tumor for high-content screening of anticancer drugs.

Additionally, inhibition or reversal of EMT is an attractive therapeutic approach that can significantly alter disease outcome. However, the bottleneck that remains for tackling this fetal health issue is that we lack of proper models that recapitulate the pathophysiology of tumor micrometastasis. A major challenge is to establish the appropriate topological interactions and spatial organization of tumor cells into clusters, aggregates, colonies and other morphological patterns. However, Current many methods are based on evenly plated cells in relatively low cell densities in 2D verses high cell density in the organism; they are under low oxygen level around 0.1-3% instead of 21% oxygen in the incubator. In tumor, the glucose level is around 5 times lower than in the culture dish and pH lactate is high while pH is low. Many study results showed that hypoglycemic/hypoxic condition in vitro mimicking the tumor microenvironment markedly reduced the efficacy of anticancer drugs [70].

To develop, to identify, and to assess in 3D tumor metastasis and invasion enables investigators to probe the basic mechanisms of morphogenesis, cell migration, cell differentiation, and recolonization at a second site and provides a future platform for testing clinically therapeutics.

Tumors are dynamic and complex structures, their composition and environment being governed by biochemical and molecular signals exchanged with the extracellular matrix. Taking this into consideration, the biological relevance of 3D tumor cell culture models [6] cannot be overemphasized.

In recent years, tissue-like models in vitro, 3D spheroids culture system were developed by employing microspheres/bio-scaffolds (Fischbach, 2007 [5]; Schneiderhan, 2007; Blanco), rotary chambers, and hanging droplets. Although useful, these techniques still pose limitations, including lack of heterogeneity due to lack of uncontrolled aggregation, and poor reproducibility due to variable size and density. Furthermore, these methods lack scalability for large experiments, especially in tumor drug screening.

Cell culture models for tumor cell invasion are currently restricted to a few widely used, potentially artificial assays (transwell invasion assays; scratch-wound migration assays). These models are not physiologically relevant.

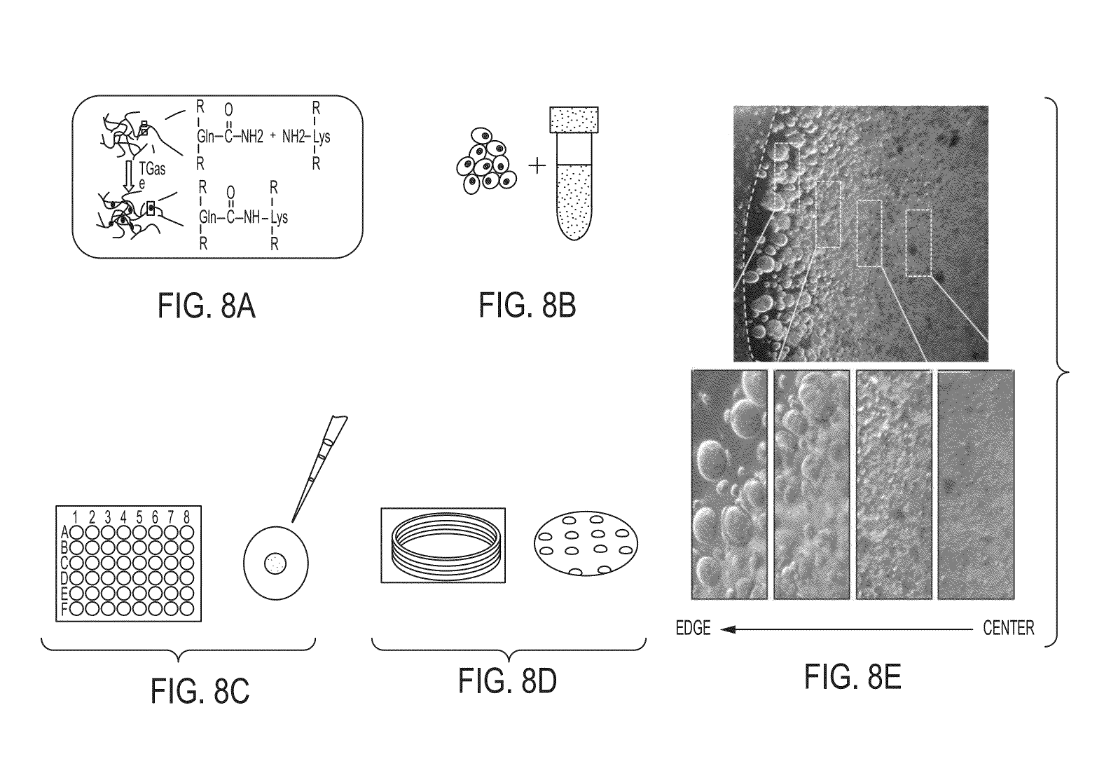

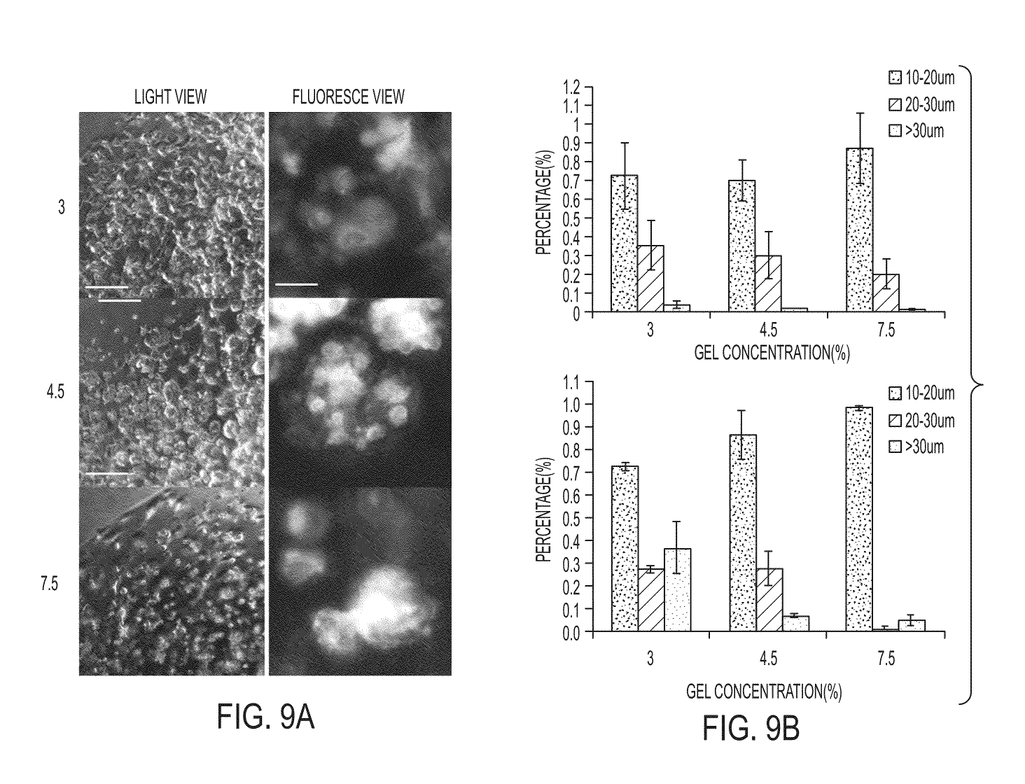

Accordingly, we have developed an injectable collagen/gelatin-based transglutaminase-crosslinked gel (Col-Tgel) system, a biologically derived matrix for cell culture and drug delivery. The biocompatibility and 3D architecture of Col-Tgel are suitable for reproducing the solid tumor microenvironment. Tumor cells cultured in Col-Tgel mimic in vivo characteristics such as tumoroid formation, matrix interaction and remodeling, responsible to metabolic gradients and mechanical restriction. Topology of matrix can be modulated through gel concentration and crosslinking rate while the stiffness of the gel can be created in the range of 10 pka-3000 pka. The gel is transparent to enable live imaging possible. Tumor cells in Col-Tgel showed cell type specific responses to 3D gel rigidity, with changes in morphology, proliferation rate, and resistance to drugs. FIG. 8 shows that colon carcinoma in the gel spontaneously formed heterogeneity phenotype to mimic human tumor. FIG. 8 shows that a 3D tumor model replicates tumor cell heterogeneous conditions. A. hydrogel formation of Tg cross-linked gelatin B. cell and gel were mixed homogeneously at room temperature and delivered as a half dome shape into single well or multiple domes in one dish (D &D). Crosslinking of gelatin tumor cells formed necrotic or apoptotic center, small to big spheroids from the innermost to the periphery region of the gel when HCT116 cells were cultured in the gel for 6 days (E). Cell density was 4×10⁶/ml of gel, each dome tumor was 204 of gel. Scale bar=1000 μm. At different depths from the surface, cells adapted different morphologies to reflect their different nutritional states and matrix topology. FIG. 9 shows that cell morphology and cluster formation is different at different gel conditions.

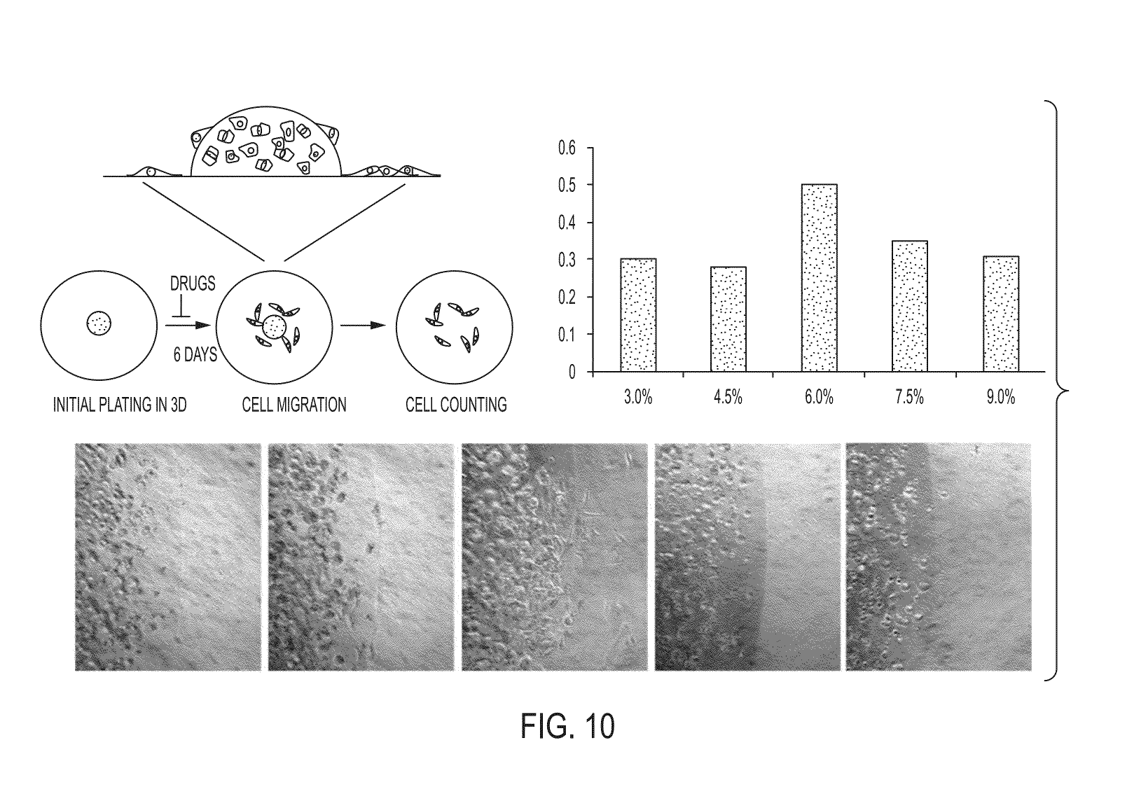

FIG. 10 shows that, for a prolonged breast cancer MDA-MD-231 culture (>6 day), cell migration was observed at the edge of the gel in the selected gel conditions.

Accordingly, one aspect of the present invention is a composition of matter comprising a substantially insoluble three-dimensional matrix for tumor modeling wherein the three-dimensional matrix comprises gelatin covalently cross-linked by the catalytic action of transglutaminase forming covalent bonds between the acyl groups of glutamine side chains and the ε-amino groups of lysine side chains. The three-dimensional matrix can comprise from about 3% to about 9% of cross-linked gelatin. Typically, the three-dimensional matrix comprises about 3%, about 4.5%, about 6%, about 7.5%, or about 9% of cross-linked gelatin. Typically, the three-dimensional matrix is in the geometrical form of a plug or a dome. Preferably, the three-dimensional matrix is in the geometrical form of a dome. The three-dimensional matrix can further comprise at least one additional protein selected from the group consisting of collagen I, collagen II, collagen III, collagen IV, laminin, vitronectin, and fibronectin. Alternatively, the three-dimensional matrix can also further comprise at least one proteoglycan. In another alternative, the three-dimensional matrix can also further comprise at least one glycoprotein. Typically, the three-dimensional matrix is substantially transparent.

Another aspect of the present invention is a tumor model comprising:

(1) a three-dimensional matrix as described above; and

(2) a tumor cell line seeded into the three-dimensional matrix.

The tumor cell line can be any conventional human or non-human tumor cell line. For example, and not by way of limitation, the tumor cell line can be breast cancer cell line MDA MB-231, breast cancer cell line MCF-7, colon cancer cell line HCT116, Saos-2 human osteocarcoma cell line, C4-2B human prostate cancer cell line, SCC-71 human oral squamous carcinoma cell line, and colon cancer cell line CFPAC-1.

The tumor model can further comprise at least one additional cell line seeded into the three-dimensional matrix, such as, but not limited to, a mesenchymal cell line.

The tumor model typically includes multicellular tumor spheroids. In the tumor model, metabolic gradients are typically present. These metabolic gradients can include, but are not limited to, a gradient of oxygen concentration so that hypoxia develops.

Yet another aspect of the present invention is a method for screening for the activity of a drug for antineoplastic activity comprising the steps of:

(1) providing a three-dimensional matrix as described above;

(2) providing a tumor cell line;

(3) seeding the tumor cell line into the three-dimensional matrix;

(4) culturing the tumor cell line in the three-dimensional matrix to provide a tumor culture in a three-dimensional environment;

(5) adding a quantity of a drug to be tested for antineoplastic activity to the tumor culture in the three-dimensional environment; and

(6) determining the effect of the drug on the viability of the tumor cell line in the three-dimensional environment to screen the drug for antineoplastic activity.

Typically, the step of determining the effect of the drug on the viability of the tumor cell line in the three-dimensional environment is performed by an assay to determine the proportion of viable cells and dead cells subsequent to the addition of the drug to the tumor culture. One method for doing this is to stain viable cells with calcein AM and to stain dead cells with ethidium homodimer-1. Other methods of determining the proportion of viable cells and dead cells, including staining methods and immunohistochemical methods, are known in the art. Alternatively, the step of determining the effect of the drug on the viability of the tumor cell line in the three-dimensional environment can be performed by determining the occurrence of apoptosis in the tumor cell line. Morphological and biochemical markers of apoptosis are known in the art and are described, for example, in M. Simboli-Campbell et al., “1,25-Dihydroxyvitamin D₃ Induces Morphological and Biochemical Markers of Apoptosis in MCF-7 Breast Cancer Cells,” J. Steroid Biochem. Mol. Biol. 58: 367-376 (1996), incorporated herein by this reference.

The drug to be tested can be a drug of any class of drugs with potential antineoplastic activity. For example, and not by way of limitation, the drug can be an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a camptothecin, an anti-tubulin agent, a thymidylate synthase inhibitor, a taxol derivative, or another class of drugs.

Yet another aspect of the present invention is a method of inducing tumor formation in an animal model for studying tumor growth in vivo comprising the steps of:

(1) providing a tumor model as described above; and

(2) implanting the tumor model into a test animal to study tumor growth in vivo.

This method can further comprise the steps of:

(3) administering a drug to be tested for antineoplastic activity into the test animal into which the tumor model has been implanted; and

(4) determining the effect of the drug on at least one parameter associated with tumor growth in the test animal.

The at least one parameter associated with tumor growth can be, but is not limited to, the rate or extent of tumor growth in the test animal as measured by tumor size or tumor burden, viability of the test animal, or activity or behavior of the test animal.

Yet another aspect of the present invention is a model for studying cell proliferation and migration. The model comprises the steps of:

(1) providing a tumor model as described above;

(2) monitoring cell proliferation within the model; and

(3) monitoring migration of cells out of the tumor model.

The rigidity of the transglutaminase gel can be controlled by gelatin concentration and cross-linking rate. Typically, cells migrate out of the tumor model driven by gel physical and chemical factors, such as rigidity, pH, O₂ level, the existence of binding sites for cell surface molecules, and other factors known in the art. The gel composite can be tailored. The cells that migrate out of the tumor model, which are not necessarily tumor cells, can migrate onto a plastic surface, or can be coated by ECM, collagen, or fibronectin. In the model, the three-dimensional structure, typically a dome, can be removed by a physical method, including, but not limited to, aspiration or scraping. The cells that have migrated out of the tumor model, which can be present on a hard surface, can be counted, or cell number can be determined by methods such as a MTT assay, a CCK-8 assay, or a bromodeoxyuridine (BrdU) assay.

Yet another aspect of the present invention is a three-dimensional stress model for assessing the existence of drug resistance. The stress can be from: (i) mechanical restriction imposed by the three-dimensional gel itself; or (ii) the buildup of at least one metabolite that results in chemical stress; the metabolite can be, but is not limited to, pH, CO₂, lactic acid, formaldehyde, NO, ROS (reactive oxygen species), RNS (reactive nitrogen species), or other stress-related metabolites. Tumor cells, in adapting to this environment, change their pattern of protein expression and turn off or turn on many signal pathways in response to the stress. Because of the pathways that are turned on as the result of the stress, drug resistance may develop. The relationship between such cancer cell stress and the development of drug resistance has been described in A. Tomida & T. Tsuruo, “Drug Resistance Mediated by Cellular Stress Response to the Microenvironment of Solid Tumors,” Anticancer Drug Res. 14: 169-177 (1999), incorporated by this reference, which describes the development of resistance to etoposide, doxorubicin, camptothecin, and vincristine. The existence of cancer cell stress, such as cancer cell stress associated with hypoxia, tends to increase the utilization of glycolysis by cancer cells at the expense of Krebs-cycle metabolism linked to oxidative phosphorylation, a phenomenon known as the Warburg effect. This may also contribute to drug resistance, as described in R.-H. Hua et al., “Inhibition of Glycolysis in Cancer Cells: A Novel Strategy to Overcome Drug Resistance Associated with Mitochondrial Respiratory Defect and Hypoxia,” Cancer Res. 65: 613-621 (2005), incorporated herein by this reference. Another mechanism that links cancer cell stress to drug resistance is the induction of glutathione S-transferases, whose catalytic activity can deactivate antineoplastic therapeutic agents, as described in J. D. Hayes & D. J. Pulford, “The Glutathione S-Transferase Supergene Family: Regulation of GST* and the Contribution of the Isoenzymes to Cancer Chemoprotection and Drug Resistance,” Crit. Rev. Biochem. Mol. Biol. 30: 445-600 (1995), incorporated herein by this reference.

Accordingly, another aspect of the present invention is a method of generating a model of stressed tumor cells and employing the model to assess drug resistance. The method comprises the steps of:

(1) providing a tumor model as described above;

(2) subjecting the tumor model to a stress selected from the group consisting of: (i) mechanical restriction; and (ii) the buildup of at least one metabolite that results in chemical stress;

(3) administering an antineoplastic therapeutic agent to the tumor model that has been subjected to the stress;

(4) determining the degree of resistance to the antineoplastic therapeutic agent administered to the tumor model that has been subjected to the stress; and

(5) comparing the degree of resistance to the antineoplastic therapeutic agent occurring in the tumor model that has been subjected to the stress to the degree of resistance to the antineoplastic therapeutic agent occurring in a culture of tumor cells that are the same as in the tumor model but that have not been subjected to stress in order to determine the degree of resistance to the antineoplastic agent induced by stress.

Typically, the degree of resistance to the antineoplastic therapeutic agent is determined by a reduction in apoptosis induced by the antineoplastic therapeutic agent, although other methods of determining drug resistance are known. For example, a fluorometric microculture cytotoxicity assay can be used, as described in R. Larsson et al., “Laboratory Determination of Chemotherapeutic Drug Resistance in Tumor Cells from Patients with Leukemia, Using a Fluorometric Microculture Cytotoxicity Assay (FMCA),” Int. J. Cancer 50: 177-185 (1992), incorporated herein by this reference.

Resistance to a wide range of antineoplastic agents can be determined by this model. The antineoplastic agent can be, but is not limited to: an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a camptothecin, an anti-tubulin agent, a thymidylate synthase inhibitor, or a taxol derivative. Particular frequently used antineoplastic agents for which resistance can be determined include taxol, etoposide, doxorubicin, camptothecin, and vincristine.

The following references are cited in this patent application by number. These references are incorporated herein by this reference. These references are not necessarily prior art:
1. Langley R R, Fidler I J (2011) The seed and soil hypothesis revisited—The role of tumor-stroma interactions in metastasis to different organs. International Journal of Cancer 128: 2527-2535.
2. Yamada K M, Cukierman E (2007) Modeling tissue morphogenesis and cancer in 3D. Cell 130: 601-610.
3. Stroock A D, Fischbach C (2010) Microfluidic culture models of tumor angiogenesis. Tissue Engineering Part A 16: 2143-2146.
4. Baker B M, Chen C S (2012) Deconstructing the third dimension—how 3D culture microenvironments alter cellular cues. Journal of Cell Science 125: 3015-3024.
5. Fischbach C, Chen R, Matsumoto T, Schmelzle T, Brugge J S, et al. (2007) Engineering tumors with 3D scaffolds. Nature methods 4: 855-860.
6. Ghajar C M, Bissell M J (2010) Tumor engineering: the other face of tissue engineering. Tissue Engineering Part A 16: 2153-2156.
7. Kim J B. Three-dimensional tissue culture models in cancer biology; 2005. Elsevier. pp. 365-377.
8. Nyga A, Cheema U, Loizidou M (2011) 3D tumour models: novel in vitro approaches to cancer studies. Journal of cell communication and signaling 5: 239-248.
9. Fischbach C, Kong H J, Hsiong S X, Evangelista M B, Yuen W, et al. (2009) Cancer cell angiogenic capability is regulated by 3D culture and integrin engagement. Proceedings of the National Academy of Sciences 106: 399-404.
10. Constantinou A I, Krygier A E, Mehta R R (1998) Genistein induces maturation of cultured human breast cancer cells and prevents tumor growth in nude mice. The American journal of clinical nutrition 68: 1426S-1430S.
11. Ong S-M, Zhao Z, Arooz T, Zhao D, Zhang S, et al. (2010) Engineering a scaffold-free 3D tumor model for in vitro drug penetration studies. Biomaterials 31: 1180-1190.
12. Timmins N E, Nielsen L K (2007) Generation of multicellular tumor spheroids by the hanging-drop method. Tissue Engineering: Springer. pp. 141-151.
13. Albini A, Iwamoto Y, Kleinman H, Martin G, Aaronson S, et al. (1987) A rapid in vitro assay for quantitating the invasive potential of tumor cells. Cancer Research 47: 3239-3245.
14. Passaniti A, Isaacs J T, Haney J A, Adler S W, Cujdik T J, et al. (1992) Stimulation of human prostatic carcinoma tumor growth in athymic mice and control of migration in culture by extracellular matrix. International Journal of Cancer 51: 318-324.
15. Miller B E, Miller F R, Heppner G H (1985) Factors affecting growth and drug sensitivity of mouse mammary tumor lines in collagen gel cultures. Cancer Research 45: 4200-4205.
16. Doillon C J, Gagnon E, Paradis R, Koutsilieris M (2004) Three-dimensional culture system as a model for studying cancer cell invasion capacity and anticancer drug sensitivity. Anticancer Research 24: 2169-2178.
17. Gurski L A, Jha A K, Zhang C, Jia X, Farach-Carson M C (2009) Hyaluronic acid-based hydrogels as 3D matrices for in vitro evaluation of chemotherapeutic drugs using poorly adherent prostate cancer cells. Biomaterials 30: 6076-6085.
18. Kibbey M C, Grant D S, Kleinman H K (1992) Role of the SIKVAV site of laminin in promotion of angiogenesis and tumor growth: an in vivo Matrigel model. Journal of the National Cancer Institute 84: 1633-1638.
19. Kessenbrock K, Plaks V, Werb Z (2010) Matrix metalloproteinases: regulators of the tumor microenvironment. Cell 141: 52-67.
20. Sung S-Y, Hsieh C-L, Wu D, Chung L W, Johnstone P A (2007) Tumor microenvironment promotes cancer progression, metastasis, and therapeutic resistance. Current problems in cancer 31: 36-100.
21. Szot C S, Buchanan C F, Freeman J W, Rylander M N (2011) 3D in vitro bioengineered tumors based on collagen I hydrogels. Biomaterials 32: 7905-7912.
22. Kuwahara K, Yang Z, Slack G C, Nimni M E, Han B (2009) Cell delivery using an injectable and adhesive transglutaminase—gelatin gel. Tissue Engineering Part C: Methods 16: 609-618.
23. Kuwahara K, Fang J Y, Yang Z, Han B (2011) Enzymatic crosslinking and degradation of gelatin as a switch for bone morphogenetic protein-2 activity. Tissue Engineering Part A 17: 2955-2964.
24. Fang J, Yang Z, Tan S, Tayag C, Nimni M E, et al. (2014) Injectable gel graft for bone defect repair. Regenerative medicine 9: 41-51.
25. Trédan O, Galmarini C M, Patel K, Tannock I F (2007) Drug resistance and the solid tumor microenvironment. Journal of the National Cancer Institute 99: 1441-1454.
26. Ungefroren H, Sebens S, Groth S, Gieseler F, Fandrich F (2011) The Src family kinase inhibitors PP2 and PP1 block TGF-beta1-mediated cellular responses by direct and differential inhibition of type I and type II TGF-beta receptors. Current Cancer Drug Targets 11: 524-535.
27. Su S, Chang Y, Andreu-Vieyra C, Fang J, Yang Z, et al. (2012) miR-30d, miR-181a and miR-199a-5p cooperatively suppress the endoplasmic reticulum chaperone and signaling regulator GRP78 in cancer. Oncogene.
28. Masood R, Kumar S R, Sinha U K, Crowe D L, Krasnoperov V, et al. (2006) EphB4 provides survival advantage to squamous cell carcinoma of the head and neck. International Journal of Cancer 119: 1236-1248.
29. Gordon E M S, Michael; Kundu, Ramendra Krishna; Han, Bo; Andrades, Jose; Nimni, Marcel; Anderson, W. French; Hall, Frederick L. (1997) Capture and Expansion of Bone Marrow-Derived Mesenchymal Progenitor Cells with a Transforming Growth Factor-β1-von Willebrand's Factor Fusion Protein for Retrovirus-Mediated Delivery of Coagulation Factor IX. Human Gene Therapy 8: 1385-1394.
30. Ahearne M, Yang Y, Liu K (2008) Mechanical characterisation of hydrogels for tissue engineering applications. Topics in tissue Engineering 4: 1-16.
31. Yang Y, Bagnaninchi P O, Ahearne M, Wang R K, Liu K-K (2007) A novel optical coherence tomography based micro-indentation technique for mechanical characterization of hydrogels. Journal of the Royal Society Interface 4: 1169-1173.
32. Chau D, Collighan R J, Verderio E A, Addy V L, Griffin M (2005) The cellular response to transglutaminase-cross-linked collagen. Biomaterials 26: 6518-6529.
33. Coligan J E, Dunn B, Ploegh H, Speicher D, Wingfield P (1996) Current protocols in protein science.
34. Davis G E, Bayless K J, Davis M J, Meininger G A (2000) Regulation of tissue injury responses by the exposure of matricryptic sites within extracellular matrix molecules. The American Journal of Pathology 156: 1489-1498.
35. Wulf E, Deboben A, Bautz F, Faulstich H, Wieland T (1979) Fluorescent phallotoxin, a tool for the visualization of cellular actin. Proceedings of the National Academy of Sciences 76: 4498-4502.
36. Tilghman R W, Blais E M, Cowan C R, Sherman N E, Grigera P R, et al. (2012) Matrix rigidity regulates cancer cell growth by modulating cellular metabolism and protein synthesis. PLoS One 7: e37231.
37. Agastin S, Giang U-B T, Geng Y, DeLouise L A, King M R (2011) Continuously perfused microbubble array for 3D tumor spheroid model. Biomicrofluidics 5: 024110.
38. Talukdar S, Mandal M, Hutmacher D W, Russell P J, Soekmadji C, et al. (2011) Engineered silk fibroin protein 3D matrices for in vitro tumor model. Biomaterials 32: 2149-2159.
39. Loessner D, Stok K S, Lutolf M P, Hutmacher D W, Clements J A, et al. (2010) Bioengineered 3D platform to explore cell—ECM interactions and drug resistance of epithelial ovarian cancer cells. Biomaterials 31: 8494-8506.
40. Bradbury P, Fabry B, O'Neill G M (2012) Occupy tissue: The movement in cancer metastasis. Cell adhesion & migration 6: 424-520.
41. Fraley S I, Feng Y, Krishnamurthy R, Kim D-H, Celedon A, et al. (2010) A distinctive role for focal adhesion proteins in three-dimensional cell motility. Nature cell biology 12: 598-604.
42. Evans C L, Abu-Yousif A O, Park Y J, Klein O J, Celli J P, et al. (2011) Killing hypoxic cell populations in a 3D tumor model with EtNBS-PDT. PLoS ONE 6: e23434.
43. Büchler P, Reber H A, Lavey R S, Tom Linson J, Büchler M W, et al. (2004) Tumor hypoxia correlates with metastatic tumor growth of pancreatic cancer in an orthotopic murine model 1. Journal of Surgical Research 120: 295-303.
44. Vaupel P, Mayer A (2007) Hypoxia in cancer: significance and impact on clinical outcome. Cancer and Metastasis Reviews 26: 225-239.
45. Brammer I, Zywietz F, Jung H (1979) Changes of histological and proliferative indices in the Walker carcinoma with tumour size and distance from blood vessel. European Journal of Cancer (1965) 15: 1329-1336.
46. Rajendran J G, Krohn K A (2005) Imaging hypoxia and angiogenesis in tumors. Radiologic Clinics of North America 43: 169-187.
47. Crescenzi V, Francescangeli A, Taglienti A (2002) New gelatin-based hydrogels via enzymatic networking. Biomacromolecules 3: 1384-1391.
48. Barcus C E, Keely P J, Eliceiri K W, Schuler L A (2013) Stiff collagen matrices increase tumorigenic prolactin signaling in breast cancer cells. Journal of Biological Chemistry 288: 12722-12732.
49. Baker E L, Lu J, Yu D, Bonnecaze R T, Zaman M H (2010) Cancer cell stiffness: integrated roles of three-dimensional matrix stiffness and transforming potential. Biophysical Journal 99: 2048-2057.
50. Hakkinen K M, Harunaga J S, Doyle A D, Yamada K M (2010) Direct comparisons of the morphology, migration, cell adhesions, and actin cytoskeleton of fibroblasts in four different three-dimensional extracellular matrices. Tissue Engineering Part A 17: 713-724.
51. Ma L, Zhou C, Lin B, Li W (2010) A porous 3D cell culture micro device for cell migration study. Biomedical microdevices 12: 753-760.
52. Zaman M H, Trapani L M, Sieminski A L, MacKellar D, Gong H, et al. (2006) Migration of tumor cells in 3D matrices is governed by matrix stiffness along with cell-matrix adhesion and proteolysis. Proceedings of the National Academy of Sciences 103: 10889-10894.
53. Paszek M J, Zahir N, Johnson K R, Lakins J N, Rozenberg G I, et al. (2005) Tensional homeostasis and the malignant phenotype. Cancer cell 8: 241-254.
54. Zahir N, Lakins J N, Russell A, Ming W, Chatterjee C, et al. (2003) Autocrine laminin-5 ligates α6β4 integrin and activates RAC and NF-κB to mediate anchorage-independent survival of mammary tumors. The Journal of cell biology 163: 1397-1407.
55. Pirazzoli V, Ferraris G M S, Sidenius N (2013) Direct evidence of the importance of vitronectin and its interaction with the urokinase receptor in tumor growth. Blood 121: 2316-2323.
56. Mao Y, Schwarzbauer J E (2005) Stimulatory effects of a three-dimensional microenvironment on cell-mediated fibronectin fibrillogenesis. Journal of cell science 118: 4427-4436.
57. Chekenya M, Hjelstuen M, Enger P Ø, Thorsen F, Jacob A L, et al. (2002) NG2 proteoglycan promotes angiogenesis-dependent tumor growth in CNS by sequestering angiostatin. The FASEB Journal 16: 586-588.
58. Hendriks J, Planelles L, de Jong-Odding J, Hardenberg G, Pals S, et al. (2005) Heparan sulfate proteoglycan binding promotes APRIL-induced tumor cell proliferation. Cell Death & Differentiation 12: 637-648.
59. Saverio B, Pierpaola D, Serenella A, Cesare C, Bruno M, et al. (2000) Tumor progression is accompanied by significant changes in the levels of expression of polyamine metabolism regulatory genes and clusterin (sulfated glycoprotein 2) in human prostate cancer specimens. Cancer Research 60: 28-34.
60. Ananthanarayanan B, Kim Y, Kumar S (2011) Elucidating the mechanobiology of malignant brain tumors using a brain matrix-mimetic hyaluronic acid hydrogel platform. Biomaterials 32: 7913-7923.
61. Bissell M J, LaBarge M A (2005) Context, tissue plasticity, and cancer: are tumor stem cells also regulated by the microenvironment? Cancer cell 7: 17.
62. Yu H, Kortylewski M, Pardoll D (2007) Crosstalk between cancer and immune cells: role of STAT3 in the tumour microenvironment. Nature Reviews Immunology 7: 41-51.
63. Allavena P, Sica A, Solinas G, Porta C, Mantovani A (2008) The inflammatory micro-environment in tumor progression: the role of tumor-associated macrophages. Critical reviews in oncology/hematology 66: 1-9.
64. Whiteside T (2008) The tumor microenvironment and its role in promoting tumor growth. Oncogene 27: 5904-5912.
65. Chaffer C L, Weinberg R A (2011) A perspective on cancer cell metastasis. Science 331: 1559-1564.
66. Condeelis J, Pollard J W (2006) Macrophages: obligate partners for tumor cell migration, invasion, and metastasis. Cell 124: 263-266.
67. Müller A, Homey B, Soto H, Ge N, Catron D, et al. (2001) Involvement of chemokine receptors in breast cancer metastasis. Nature 410: 50-56.
68. Brabletz, T., F. Hlubek, et al. (2005). Invasion and metastasis in colorectal cancer: epithelial-mesenchymal transition, mesenchymal-epithelial transition, stem cells and [symbol] -catenin. Cells Tissues Organs 179(1-2): 56-65.
69. Friedl, P. and K. Wolf (2010). Plasticity of cell migration: a multiscale tuning model. The Journal of cell biology 188(1): 11-19.
70. Onozuka, H., K. Tsuchihara, et al. (2011). Hypoglycemic/hypoxic condition in vitro mimicking the tumor microenvironment markedly reduced the efficacy of anticancer drugs. Cancer science 102(5): 975-982.
71. Petrie, R. J., N. Gavara, et al. (2012). Nonpolarized signaling reveals two distinct modes of 3D cell migration. The Journal of cell biology 197(3): 439-455.
72. Tsai, J. H., Donaher, J. L. et al. (2012). Spatiotemporal regulation of epithelial-mesenchymal transition is essential for squamous cell carcinoma metastasis. Cancer cell.
73. Wyckoff, J. B., S. E. Pinner, et al. (2006). ROCK- and Myosin-Dependent Matrix Deformation Enables Protease-Independent Tumor-Cell Invasion In Vivo. Current Biology 16(15): 1515-1523.
74. Yang, M.-H., M.-Z. Wu, et al. (2008). Direct regulation of TWIST by HIF-1 [symbol] promotes metastasis.” Nature cell biology 10(3): 295-305.
75. Chaffer, C. L., Thompson, E. W., et al. (2006). Mesenchymal and Epithelial Transition in Development and Disease. Cells Tissues Organs 185: 7-19.
76. Korpal, M., Eli, B. J., et al. (2011). Direct Targeting of Sec23 by mi-R200s Influences Cancer Cell Secretome and Promotes Metastatic Colonization. Nature Medicine 17(9): 1101-1108.

### ADVANTAGES OF THE INVENTION

The present invention provides an improved three-dimensional tumor model that allows a researcher or clinician to study tumor formation, growth, and treatment in vitro in a model that preserves many of the interactions between tumor cells and between tumor cells and neighboring non-malignant cells such as mesenchymal cells that occur in vivo. The tumor model can be used to screen potential antineoplastic drugs and to implant tumors in vivo. The tumor model can also be used to assay resistance to antineoplastic drugs resulting from stress to tumor cells.

Compositions according to the present invention possess industrial applicability as compositions of matter for the preparation of tumor models. Methods according to the present invention possess industrial applicability as in vitro screening methods or methods for tumor implantation into non-human test animals, as well as methods for determining and measuring tumor migration.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A composition of matter comprising a substantially insoluble three-dimensional matrix for tumor modeling wherein the three-dimensional matrix comprises gelatin covalently cross-linked by the catalytic action of transglutaminase forming covalent bonds between the acyl groups of glutamine side chains and the ε-amino groups of lysine side chains.

2. The composition of matter of claim 1 wherein the three-dimensional matrix comprises from about 3% to about 9% of cross-linked gelatin.

3. The composition of matter of claim 2 wherein the three-dimensional matrix comprises about 3%, about 4.5%, about 6%, about 7.5%, or about 9% of cross-linked gelatin.

4. The composition of matter of claim 1 wherein the three-dimensional matrix is in the geometrical form of a plug or a dome.

5. The composition of matter of claim 4 wherein the three-dimensional matrix is in the geometrical form of a dome.

6. The composition of matter of claim 1 wherein the three-dimensional matrix further comprises at least one additional protein selected from the group consisting of collagen I, collagen II, collagen III, collagen IV, laminin, vitronectin, and fibronectin.

7. The composition of matter of claim 1 wherein the three-dimensional matrix further comprises at least one proteoglycan.

8. The composition of matter of claim 1 wherein the three-dimensional matrix further comprises at least one glycoprotein.

9. The composition of matter of claim 1 wherein the three-dimensional matrix is substantially transparent.

10. A tumor model comprising:
(a) the three-dimensional matrix of claim 1; and
(b) a tumor cell line seeded into the three-dimensional matrix.

11. The tumor model of claim 10 wherein the three-dimensional matrix comprises from about 3% to about 9% of cross-linked gelatin.

12. The tumor model of claim 11 wherein the three-dimensional matrix comprises about 3%, about 4.5%, about 6%, about 7.5%, or about 9% of cross-linked gelatin.

13. The tumor model of claim 10 wherein the three-dimensional matrix is in the geometrical form of a plug or a dome.

14. The tumor model of claim 13 wherein the three-dimensional matrix is in the geometrical form of a dome.

15. The tumor model of claim 10 wherein the three-dimensional matrix further comprises at least one additional protein selected from the group consisting of collagen I, collagen II, collagen III, collagen IV, laminin, vitronectin, and fibronectin.

16. The tumor model of claim 10 wherein the three-dimensional matrix further comprises at least one proteoglycan.

17. The tumor model of claim 10 wherein the three-dimensional matrix further comprises at least one glycoprotein.

18. The tumor model of claim 10 wherein the three-dimensional matrix is substantially transparent.

19. The tumor model of claim 10 wherein the tumor cell line is selected from the group consisting of breast cancer cell line MDA MB-231, breast cancer cell line MCF-7, colon cancer cell line HCT116, Saos-2 human osteocarcoma cell line, C4-2B human prostate cancer cell line, SCC-71 human oral squamous carcinoma cell line, and colon cancer cell line CFPAC-1.

20. The tumor model of claim 10 wherein the tumor model further comprises at least one additional cell line seeded into the three-dimensional matrix.

21. The tumor model of claim 20 wherein the at least one additional cell line is a mesenchymal cell line.

22. The tumor model of claim 10 wherein the tumor model includes multicellular tumor spheroids.

23. The tumor model of claim 10 wherein metabolic gradients are present.

24. The tumor model of claim 23 wherein the metabolic gradients include a gradient of oxygen concentration so that hypoxia develops.

25. A method of screening for the activity of a drug for antineoplastic activity comprising the steps of:
(a) providing the three-dimensional matrix of claim 1;
(b) providing a tumor cell line;
(c) seeding the tumor cell line into the three-dimensional matrix;
(d) culturing the tumor cell line in the three-dimensional matrix to provide a tumor culture in a three-dimensional environment;
(e) adding a quantity of a drug to be tested for antineoplastic activity to the tumor culture in the three-dimensional environment; and
(f) determining the effect of the drug on the viability of the tumor cell line in the three-dimensional environment to screen the drug for antineoplastic activity.

26. The method of claim 25 wherein the step of determining the effect of the drug on the viability of the tumor cell line in the three-dimensional environment is performed by an assay to determine the proportion of viable cells and dead cells subsequent to the addition of the drug to the tumor culture.

27. The method of claim 26 wherein the assay to determine the proportion of viable cells and dead cells subsequent to the addition of the drug to the tumor culture is performed by staining viable cells with calcein AM and staining dead cells with ethidium homodimer-1.

28. The method of claim 25 wherein the step of determining the effect of the drug on the viability of the tumor cell line in the three-dimensional environment is performed by determining the occurrence of apoptosis in the tumor cell line.

29. The method of claim 25 wherein the drug to be tested for antineoplastic activity is selected from the group consisting of an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a camptothecin, an anti-tubulin agent, a thymidylate synthase inhibitor, a taxol derivative.

30. A method of generating a model of stressed tumor cells and employing the model to assess drug resistance comprising the steps of:
(a) providing the tumor model of claim 10;
(b) subjecting the tumor model to a stress selected from the group consisting of: (i) mechanical restriction; and (ii) the buildup of at least one metabolite that results in chemical stress;
(c) administering an antineoplastic therapeutic agent to the tumor model that has been subjected to the stress;
(d) determining the degree of resistance to the antineoplastic therapeutic agent administered to the tumor model that has been subjected to the stress; and
(e) comparing the degree of resistance to the antineoplastic therapeutic agent occurring in the tumor model that has been subjected to the stress to the degree of resistance to the antineoplastic therapeutic agent occurring in a culture of tumor cells that are the same as in the tumor model but that have not been subjected to stress in order to determine the degree of resistance to the antineoplastic agent induced by stress.

31. The method of claim 30 wherein the degree of resistance to the antineoplastic therapeutic agent is determined by a reduction in apoptosis induced by the antineoplastic therapeutic agent.

32. The method of claim 30 wherein the antineoplastic therapeutic agent is selected from the group consisting of: an alkaloid, an antimetabolite, a nucleoside or nucleotide analog, a topoisomerase inhibitor, a platinum compound, an antibiotic, a VEGF inhibitor, a tyrosine kinase inhibitor, a monoclonal antibody, a cam ptothecin, an anti-tubulin agent, a thymidylate synthase inhibitor, and a taxol derivative.

33. The method of claim 30 wherein the antineoplastic agent is selected from the group consisting of taxol, etoposide, doxorubicin, camptothecin, and vincristine.
